(19) United States
(12) Patent Application Publication
(10) Pub. No.: US 2006/0281726 A1
(43) Pub. Date: Dec. 14, 2006

(54) Piperidine derivatives for the treatment of chemokine or h1 mediated disease state

(76) Inventors: Christopher Luckhurst, Leicestershire (GB); Matthew Perry, Leicestershire (GB); Brian Springthorpe, Leicestershire (GB)

Correspondence Address: FISH & RICHARDSON P.C., P.O BOX 1022, MINNEAPOLIS, MN, 55440-1022, US

(21) Appl. No.: 10/549,868

(22) PCT Filed: Mar. 23, 2004
(86) PCT No.: PCT/SE04/00450

§ 371 Date: Aug. 17, 2006

(30) Foreign Application Priority Data
Mar. 25, 2003 (SE) 0300850-5

(51) Int. Cl. C07D 401/14; C07D 401/02; A61K 31/4545; A61K 31/5377; A61K 31/55; A61K 31/496; C07D 413/14

(52) U.S. Cl. 514/210.2; 514/316; 546/187; 546/188; 546/189; 514/217.04; 514/235.2; 514/253.01; 544/128; 544/364; 540/597

(57) ABSTRACT

The present invention provides a compound of a formula (I), wherein the variables are defined herein; to a process for preparing such a compound; and to the use of such a compound in the treatment of a chemokine (such as CCR3) or H1 mediated disease state.

The present invention concerns piperidine derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in WO99/38514,WO99/04794 and WO00/35877.

Histamine is a basic amine, 2-(4-imidazolyl)-ethylamine, and is formed from histidine by histidine decarboxylase. It is found in most tissues of the body, but is present in high concentrations in the lung, skin and in the gastrointestinal tract. At the cellular level inflammatory cells such as mast cells and basophils store large amounts of histamine. It is recognised that the degranulation of mast cells and basophils and the subsequent release of histamine is a fundamental mechanism responsible for the clinical manifestation of an allergic process. Histamine produces its actions by an effect on specific histamine G-protein coupled receptors, which are of three main types, H1, H2 and H3. Histamine H1 antagonists comprise the largest class of medications used in the treatment of patients with allergic disorders, especially rhinitis and urticaria. H1 antagonists are useful in controlling the allergic response by for example blocking the action of histamine on post-capillary venule smooth muscle, resulting in decreased vascular permeability, exudation and oedema. The antagonists also produce blockade of the actions of histamine on the H1 receptors on c-type nociceptive nerve fibres, resulting in decreased itching and sneezing.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or α) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

Viral infections are known to cause lung inflammation. It has been shown experimentally that the common cold increases mucosal output of eotaxin in the airways. Instillation of eotaxin into the nose can mimic some of the signs and symptoms of a common cold. (See, Greiff L et al Allergy (1999) 54(11) 1204-8 [Experimental common cold increase mucosal output of eotaxin in atopic individuals] and Kawaguchi M et al Int. Arch. Allergy Immunol. (2000) 122 S1 44 [Expression of eotaxin by normal airway epithelial cells after virus A infection].)

The present invention provides a compound of formula (I): 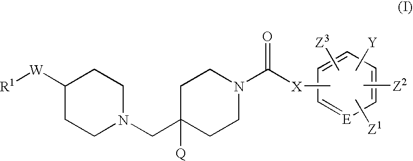
wherein:
E is CH or N;
Q is hydrogen or hydroxy;
W is CH₂, O or NR²;
X is a bond, CH₂ or CH₂O;
Y is OH, CO₂R³, SO₃H, CH₂CO₂R³, CH₂SO₃H, OCH₂CO₂R³ or OCH₂SO₃H;
Z¹, Z², Z³ are, independently, hydrogen, halogen, cyano, nitro, hydroxy, NR⁴R^{5,} C₁₋₆ alkyl (optionally substituted with halogen), C₁₋₆ alkoxy (optionally substituted with halogen), S(O)ₚ(C₁₋₆ alkyl), S(O)_{q}CF₃ or S(O)₂NR⁶R⁷;
R¹ is phenyl optionally substituted by halogen, cyano, C₁₋₄ alkyl, C₁₋₄ haloalkyl, C₁₋₄ alkoxy or C₁₋₄ haloalkoxy;
R² is hydrogen or C₁₋₄ alkyl;
R³ is hydrogen, C₁₋₆ alkyl or benzyl;
p and q are, independently, 0, 1 or 2;
R⁴, R⁵, R⁶ and R⁷ are, independently, hydrogen, C₁₋₆ alkyl (optionally substituted by halogen, hydroxy or C₃₋₁₀ cycloalkyl), CH₂(C₂₋₅ alkenyl), phenyl (itself optionally substituted by halogen, hydroxy, nitro, NH₂, NH(C₁₋₄ alkyl), N(C₁₋₄ alkyl)₂ (and these alkyl groups may join to form a ring as described for R⁴ and R⁵ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂ (and these alkyl groups may join to form a ring as described for R⁴ and R⁵ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
   alternatively NR⁴R⁵ or NR⁶R⁷ may, independently, form a 4-7 membered heterocyclic ring, azetidine, pyrrolidine, piperidine, azepine, morpholine or piperazine, the latter optionally substituted by C₁₋₄ alkyl on the distal nitrogen; or an N-oxide thereof; or a pharmaceutically acceptable salt thereof; or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, dihydrochloride, hydrobromide, phosphate, sulfate, acetate, diacetate, fumarate, maleate, tartrate, citrate, oxalate, methanesulfonate or p-toluenesulfonate.

When the compound of formula (I) comprises an acid (for example a carboxylic acid) and/or phenolic group the invention includes salts of such groups. Suitable salts of such groups include alkali metal or alkaline earth metal salts such as salts with sodium, potassium, magnesium or calcium.

The compounds of the invention may exist as solvates (such as hydrates) and the present invention covers all such solvates.

Halogen includes fluorine, chlorine, bromine and iodine. Halogen is, for example, fluorine or chlorine.

Alkyl groups and moieties are straight or branched chain and comprise, for example, 1 to 6 (such as 1 to 4) carbon atoms. Examples of alkyl groups are methyl, ethyl, n-propyl, iso-propyl or tert-butyl.

Haloalkyl groups and moieties comprise an alkyl part, as defined above, and one or more (for example 1 to 6) of the same or different halogen atoms. Haloalkyl is, for example, CF₃.

Alkenyl groups comprise, for example, 2 to 6 (such as 2 to 4) carbon atoms. Examples of alkenyl groups are vinyl or allyl.

In one embodiment cycloalkyl groups comprise from 3 to 10 (such as 3 to 8, for example 3 to 6) carbon atoms and are mono-, bi or tricyclic. Cycloalkyl is, for example, cyclopropyl, cyclopentyl, cyclohexyl, norbornyl or camphoryl. The cycloalkyl ring is optionally fused to a benzene ring (for example forming a bicyclo[4.2.0]octa-1,3,5-trienyl or indanyl ring system). In a further embodiment cycloalkyl is monocyclic.

Heterocyclyl is an aromatic or non-aromatic 5 or 6 membered ring, optionally fused to one or more other rings, comprising at least one heteroatom selected from the group comprising nitrogen, oxygen and sulfur; or an N-oxide thereof, or an S-oxide or S-dioxide thereof. Heterocyclyl is, for example, furyl, thienyl (also known as thiophenyl), pyrrolyl, 2,5-dihydropyrrolyl, thiazolyl, pyrazolyl, oxazolyl, isoxazolyl, imidazolyl, piperidinyl, morpholinyl, pyridinyl, dihydropyridinyl (for example in a 6-oxo-1,6-dihydro-pyridinyl moiety), pyrimidinyl, indolyl, 2,3-dihydroindolyl, benzo[b]furyl (also known as benzfuryl), benz[b]thienyl (also known as benzthienyl or benzthiophenyl), 2,3-dihydrobenz[b]thienyl (for example in a 1-dioxo-2,3-dihydrobenz[b]thienyl moiety), indazolyl, benzimidazolyl, benztriazolyl, benzoxazolyl, benzthiazolyl (for example in a 1H-benzthiazol-2-one-yl moiety), 2,3-dihydrobenzthiazolyl (for example in a 2,3-dihydrobenzthiazol-2-one-yl moiety), 1,2,3-benzothiadiazolyl, an imidazopyridinyl (such as imidazo[1,2a]pyridinyl), thieno[3,2-b]pyridin-6-yl, 1,2,3-benzoxadiazolyl, benzo[1,2,3]thiadiazolyl, 2,1,3-benzothiadiazolyl, benzofurazan (also known as 2,1,3-benzoxadiazolyl), quinoxalinyl, dihydro-1-benzopyryliumyl (for example in a coumarinyl or a chromonyl moiety), 3,4-dihydro-1H-2,1-benzothiazinyl (for example in a 2-dioxo-3,4-dihydro-1H-2,1-benzothiazinyl moiety), a pyrazolopyridine (for example 1H-pyrazolo[3,4-b]pyridinyl), a purine (for example in a 3,7-dihydro-purin-2,6-dione-8-yl moiety), quinolinyl, isoquinolinyl, dihydroisoquinolinyl (for example in a 2H-isoquinolin-1-one-yl moiety), a naphthyridinyl (for example [1,6]naphthyridinyl or [1,8]naphthyridinyl), a dihydro[1,8]naphthyridinyl (for example in a 1H-[1,8]naphthyridin-4-one-yl moiety), a benzothiazinyl, a dihydrobenzothiazinyl (for example in a 4H-benzo[1,4]thiazin-3-one-yl moiety), benzo[d]imidazo[2,1-b]thiazol-2-yl or dibenzothiophenyl (also known as dibenzothienyl); or an N-oxide thereof, or an S-oxide or S-dioxide thereof.

An N-oxide of a compound of formula (I) is, for example, a 1-oxy-[1,4′]bipiperidinyl-1′-yl compound.

Heterocyclyl is, for example, pyrimidinyl or pyridinyl. In a further aspect of the invention heterocyclyl is optionally substituted by C₁₋₄ alkyl or C₁₋₄ alkoxy.

In one particular aspect the invention provides a compound of formula (I), wherein E is CH; Q is hydrogen or hydroxy; W is CH₂, O or NR²; X is a bond, CH₂ or CH₂O; Y is OH, CO₂R³, SO₃H, CH₂CO₂R³, CH₂SO₃H, OCH₂CO₂R³ or OCH₂SO₃H; Z¹, Z², Z³ are, independently, hydrogen, halogen, cyano, nitro, hydroxy, NR⁴R⁵, C₁₋₆ alkyl (optionally substituted with halogen), C₁₋₆ alkoxy (optionally substituted with halogen), S(O)ₚ(C₁₋₆ alkyl), S(O)_{q}CF₃ or S(O)₂NR⁶R⁷; R¹ is phenyl optionally substituted by halogen, cyano, C₁₋₄ alkyl, C₁₋₄ haloalkyl, C₁₋₄ alkoxy or C₁₋₄ haloalkoxy; R² is hydrogen or C₁₋₄ alkyl; R³ is hydrogen, C₁₋₆ alkyl or benzyl; p and q are, independently, 0, 1 or 2; R⁴, R⁵, R⁶ and R⁷ are, independently, hydrogen, C₁₋₆ alkyl (optionally substituted by halogen, hydroxy or C₃₋₁₀ cycloalkyl), CH₂(C₂₋₅ alkenyl), phenyl (itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃); alternatively NR⁴R⁵ or NR⁶R⁷ may, independently, form a 4-7 membered heterocyclic ring, azetidine, pyrrolidine, piperidine, azepine, morpholine or piperazine, the latter optionally substituted by C₁₋₄ alkyl on the distal nitrogen; or an N-oxide thereof; or a pharmaceutically acceptable salt thereof; or a solvate thereof.

In another aspect the invention provides a compound of formula (I) wherein W is O.

In a further aspect the invention provides a compound of formula (I) wherein E is CH.

In yet another aspect R¹ is phenyl optionally substituted (for example independently mono- or di-substituted) with halogen (for example chlorine or fluorine), C₁₋₄ alkyl (for example methyl) or C₁₋₄ alkoxy (for example methoxy).

In a further aspect R¹ is phenyl optionally substituted (for example with one, two or three of the same or different) with fluorine, chlorine, C₁₋₄ alkyl (for example methyl) or C₁₋₄ alkoxy (for example methoxy). In a still further aspect R¹ is phenyl substituted by one, two or three (for example two or three) substituents independently selected from: fluorine, chlorine and methyl. For example R¹ is 3,4-dichlorophenyl, 2,4-dichloro-3-methylphenyl, 3,4-dichloro-2-methylphenyl, 2,4-dichlorophenyl, 4-chloro-2-methylphenyl or 2-chloro4-fluorophenyl.

In a still further aspect of the invention Q is hydrogen.

In another aspect of the invention X is a bond.

In yet another aspect of the invention R³ is hydrogen or C₁₋₄ alkyl (such as methyl). In another aspect R³ is hydrogen.

In a further aspect of the invention Y is CO₂H, CO₂(C₁₋₄ alkyl), CH₂CO₂H or OH. In a still further aspect of the invention Y is CO₂H.

In another aspect of the invention Y is ortho to X.

In a further aspect of the invention Z¹, Z² and Z³ are, independently, hydrogen, halogen, cyano, C₁₋₄ alkyl (such as methyl or ethyl), C₁₋₄ alkoxy (such as methoxy or ethoxy), CF₃, OCF₃, S(O)₂(C₁₋₄ alkyl) (such as S(O)₂CH₃) or S(O)₂NH₂.

In another aspect of the invention Z¹ is hydrogen, halogen (such as chloro or fluoro), C₁₋₄ alkyl (such as methyl or ethyl), C₁₋₄ alkoxy (such as methoxy or ethoxy), OH or S(O)₂(C₁₋₄ alkyl) (such as S(O)₂CH₃).

In yet another aspect of the invention Z² is hydrogen or halogen (such as chloro or fluoro).

In a further aspect of the invention Z³ is hydrogen.

In a still further aspect the present invention provides a compound of formula (I) wherein: E is CH; Q is hydrogen; W is O; X is a bond; Y is CO₂H, CO₂(C₁₋₄ alkyl), CH₂CO₂H or OH; Z¹ is hydrogen, halogen (such as chloro or fluoro), C₁₋₄ alkyl (such as methyl or ethyl), C₁₋₄ alkoxy (such as methoxy or ethoxy), OH or S(O)₂(C₁₋₄ alkyl) (such as S(O)₂CH₃); Z² is hydrogen or halogen (such as chloro or fluoro); Z³ is hydrogen; and R¹ is phenyl substituted by halogen (for example by one or two chlorine atoms) or C₁₋₄ alkyl (for example methyl); or a pharmaceutically acceptable salt thereof.

In another aspect the present invention provides a compound of formula (I) wherein: E is CH; Q is hydrogen; W is O; X is a bond; Y is CO₂H; Z¹, Z² and Z³ are, independently, hydrogen, hydroxy or S(O)₂(C₁₋₄ alkyl) (for example S(O)₂CH₃); and R¹ is phenyl substituted by halogen (for example by one or two chlorine atoms) or C₁₋₄ alkyl (for example methyl).

The compounds of the present invention can be prepared as described below.

A compound of formula (I), wherein Y is CO₂H, CH₂CO₂H or OCH₂CO₂H said Y group being ortho to the group X, can be prepared by acylating a compound of formula (II): 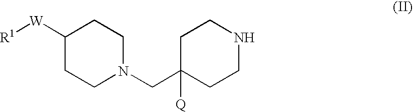
via the ring opening of an anhydride of formula (III): 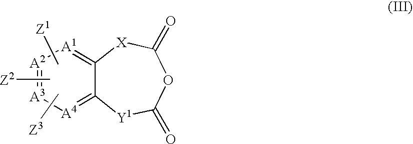
wherein one of A¹, A², A³ and A⁴ is CH or N; the other three of A¹, A², A³ and A⁴ carbon and each of the three carries Z¹, Z² or Z³, there being only one of each of Z³; X is as defined above; and Y¹ is a bond, CH₂ or OCH₂; in the presence of a tertiary amine (such as triethylamine), in a suitable solvent (such as acetonitrile) elevated temperature (such as in the range 60-100° C.).

Alternatively, a compound of formula (I), wherein Y is CO₂R³, CH₂CO₂H OCH₂CO₂R³ and R³ is not hydrogen, can be prepared by coupling a compound of (II) with a compound of formula (IV): 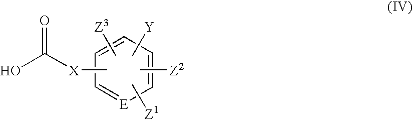
either going via the acid chloride of the compound of formula (IV) (using standard techniques) or by using a coupling reagent (such as PyBrOP or HATU) under su conditions known in the art.

A compound of formula (I), Wherein X is a bond and Y is CO₂R³, can be by carbonylation (such as palladium catalysed carbonylation) of a compound of (V): 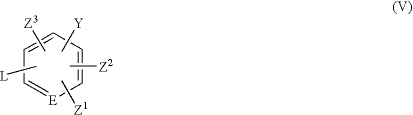
wherein L is chloro, bromo, iodo or O-triflate, and then quenching the product with a compound of formula (II).

A compound of formula (I), wherein X is a bond, Y is CO₂R³, R³ is not and R¹ does not have a chloro, bromo or iodo substituent, can also be made by compound of formula (II) with an acid of formula (VI): 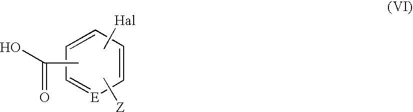
wherein Hal is chloro, bromo or iodo, under the coupling conditions described above; then carbonylating the compound so formed (such as using a palladium catalysed carbonylation); and then quenching the product so formed with a C₁₋₆ aliphatic alcohol or benzylalcohol.

For a compound of formula (I) where Y is or includes a CO₂R³ group:

when R³ is hydrogen said compound can be converted to a compound of the invention where R³ is not hydrogen by a standard esterification method well known in the art; and,

when R³ is not hydrogen said compound can be converted to a compound of the invention where R³ is hydrogen by a standard ester hydrolysis method well known in the art.

A compound of formula (II) can be prepared by deprotecting a compound of formula (VII): 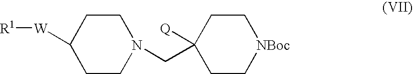
for example using trifluoroacetic acid in a suitable solvent (such as dichloromethane) or using a source of hydrogen chloride in a suitable solvent (such as dioxane).

A compound of formula (VII), wherein Q is hydrogen, can be prepared by reacting a compound of formula (VIII): 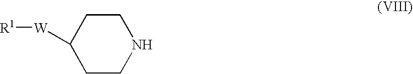
with a compound of formula (IX): 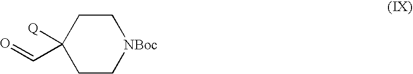
in the presence of NaBH(OAc)₃ and acetic acid, in a suitable solvent (such as tetrahydrofuran or dichloromethane).

A compound of formula (VI), wherein Q is hydroxy, can be prepared by reacting a compound of formula (VIII) with a compound of formula (X): 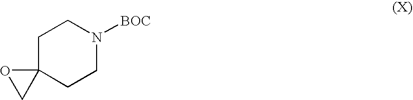
in a suitable solvent (such as a C₁₋₆ aliphatic alcohol, for example ethanol) at room temperature.

The preparation of various intermediates can be found in WO00/66559 and WO01/77101; alternatively they can be prepared by using or adapting literature methods.

Further compounds of formula (I) can be prepared by adaptation of: the routes described above, methods described in the art or the Examples recited below.

Compounds of formula (II) to (X) can be prepared by using or adapting methods described in the art. The preparation of various phenoxy piperidines is described in WO 01/77101.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of chemokine receptor (especially CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these conditions are:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata, corneal ulcer or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), peridontal disease, Sezary syndrome, idiopathic thrombocytopenia pupura or disorders of the menstrual cycle.

The compounds of formula (I) or a pharmaceutically acceptable salt thereof or a solvate thereof, are also H1 antagonists (and can, therefore, be used in the treatment of allergic disorders); and may also be used to control a sign and/or symptom of what is commonly referred to as a cold (for example a sign and/or symptom of a common cold or influenza or other associated respiratory virus infection).

According to a further feature of the present invention there is provided a method for treating a chemokine mediated disease state (especially a CCR3 mediated disease state) in a mammal, such as man, suffering from, or at risk of, said disease state, which comprises administering to a mammal in need of such treatment a therapeutically effective amount of a compound of the formula (I) or a pharmaceutically acceptable salt thereof or a solvate thereof.

According to another feature of the present invention there is provided a method for antagonising H1 in a mammal, such as man, suffering from, or at risk of, an H1 mediated disease state, which comprises administering to a mammal in need of such treatment a therapeutically effective amount of a compound of the formula (I) or a pharmaceutically acceptable salt thereof or a solvate thereof.

According to yet another feature of the present invention there is provided a method for treating a sign and/or symptom of what is commonly referred to as a cold in a mammal, such as man, suffering from, or at risk of, said disease state, which comprises administering to a mammal in need of such treatment a therapeutically effective amount of a compound of the formula (I) or a pharmaceutically acceptable salt thereof or a solvate thereof.

The invention also provides a compound of the formula (I), or a pharmaceutically acceptable salt thereof or a solvate thereof, for use in therapy.

In another aspect the invention provides the use of a compound of formula (I), or a pharmaceutically acceptable salt thereof or a solvate thereof, in the manufacture of a medicament for use in therapy (for example modulating chemokine receptor activity (especially CCR3 receptor activity), antagonising H1 or treating a sign and/or symptom of what is commonly referred to as a cold).

The invention further provides the use of a compound of formula (I), or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata, corneal ulcer or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle; in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of formula (I), or a pharmaceutically acceptable salt thereof, is useful in the treatment of asthma.

The present invention also provides a the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In order to use a compound of the invention, or a pharmaceutically acceptable salt thereof or solvate thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof or a solvate thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.01 mgkg⁻¹ to 100 mgkg⁻¹, preferably in the range of 0.1 mgkg⁻¹ to 20 mgkg⁻¹, of the active ingredient administered, for example, 1 to 4 times per day.

The invention further relates to combination therapies wherein a compound of formula (1) or a pharmaceutically acceptable salt, solvate or in vivo hydrolysable ester thereof, or a pharmaceutical composition or formulation comprising a compound of formula (1) is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents such as: Non-steroidal anti-inflammatory agents (hereinafter NSAIDs) including non-selective cyclo-oxygenase (COX)-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate, leflunomide; hydroxychloroquine, d-penicillamine, auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab , and CDP-870) and TNF receptor antagonists including immunoglobulin molecules (such as etanercept) and low-molecular-weight agents such as pentoxyfylline.

The present invention still further relates to the combination of a compound of the invention together with modulators of chemokine receptor function such as antagonists of CCR1, CCR2, CCR2A, CCR2B, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; N-(5-substituted)-thiophene-2-alkylsulfonamides; 2,6-di-tert-butylphenolhydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a phosphodiesterase (PDE) inhibitor such as the methylxanthanines including theophylline and aminophylline; and selective PDE isoenzyme inhibitors including PDE4 inhibitors and inhibitors of the isoform PDE4D, and inhibitors of PDE5.

The present invention still further relates to the combination of a compound of the invention together with histamine type 1 receptor antagonists such as cetirizine, loratadine, desloratadine, fexofenadine, acrivastine, terfenadine, astemizole, azelastine, levocabastine, chlorpheniramine, promethazine, cyclizine, and mizolastine applied orally, topically or parenterally.

The present invention still further relates to the combination of a compound of the invention together with a proton pump inhibitor (such as omeprazole) or gastroprotective histamine type 2 receptor antagonist.

The present invention still further relates to the combination of a compound of the invention with antagonists of the histamine type 4 receptor.

The present invention still further relates to the combination of a compound of the invention together with an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride, and ethylnorepinephrine hydrochloride.

The present invention still further relates to the combination of a compound of the invention together with anticholinergic agents including muscarinic receptor (M1, M2, and M3) antagonists such as atropine, hyoscine, glycopyrrrolate, ipratropium bromide, tiotropium bromide, oxitropium bromide, pirenzepine, and telenzepine.

The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol , including chiral enantiomers thereof.

The present invention still further relates to the combination of a compound of the invention together with a chromone, including sodium cromoglycate and nedocromil sodium.

The present invention still further relates to the combination of a compound of the invention together with a glucocorticoid, such as flunisolide, triamcinolone acetonide, beclomethasone dipropionate, budesonide, fluticasone propionate, ciclesonide, and mometasone furoate.

The present invention still further relates to the combination of a compound of the invention together with an agent that modulate nuclear hormone receptors such as PPARs.

The present invention still further relates to the combination of a compound of the invention together with an immunoglobulin (Ig) or Ig preparation or an antagonist or antibody modulating Ig function such as anti-IgE (e.g. omalizumab).

The present invention still further relates to the combination of a compound of the invention together with other systemic or topically-applied anti-inflammatory agents including thalidomide and derivatives, retinoids, dithranol, and calcipotriol.

The present invention still further relates to the combination of a compound of the invention together with combinations of aminosalicylates and sulfapyridine such as sulfasalazine, mesalazine, balsalazide, and olsalazine; and immunomodulatory agents such as the thiopurines, and corticosteroids such as budesonide.

The present invention still further relates to the combination of a compound of the invention together with an antibacterial agent including penicillin derivatives, tetracyclines, macrolides, beta-lactams, fluoroquinolones, metronidazole, and inhaled aminoglycosides; and antiviral agents including acyclovir, famciclovir, valaciclovir, ganciclovir, cidofovir; amantadine, rimantadine; ribavirin; zanamavir and oseltamavir; protease inhibitors such as indinavir, nelfinavir, ritonavir, and saquinavir; nucleoside reverse transcriptase inhibitors such as didanosine, lamivudine, stavudine, zalcitabine, zidovudine; non-nucleoside reverse transcriptase inhibitors such as nevirapine, efavirenz.

The present invention still further relates to the combination of a compound of the invention together with cardiovascular agents such as calcium channel blockers, beta-adrenoceptor blockers, angiotensin-converting enzyme (ACE) inhibitors, angiotensin-2 receptor antagonists; lipid lowering agents such as statins, and fibrates; modulators of blood cell morphology such as pentoxyfylline; thrombolytics, and anticoagulants including platelet aggregation inhibitors.

The present invention still further relates to the combination of a compound of the invention together with CNS agents such as antidepressants (such as sertraline), anti-Parkinsonian drugs (such as deprenyl, L-dopa, ropinirole, pramipexole, MAOB inhibitors such as selegine and rasagiline, comP inhibitors such as tasmar, A-2 inhibitors, dopamine reuptake inhibitors, NMDA antagonists, nicotine agonists, dopamine agonists and inhibitors of neuronal nitric oxide synthase), and anti-Alzheimer's drugs such as donepezil, rivastigmine, tacrine, COX-2 inhibitors, propentofylline or metrifonate.

The present invention still further relates to the combination of a compound of the invention together with agents for the treatment of acute and chronic pain, including centrally and peripherally-acting analgesics such as opioid analogues and derivatives, carbamazepine, phenytoin, sodium valproate, amitryptiline and other antidepressant agents, paracetamol, and non-steroidal anti-inflammatory agents.

The present invention still further relates to the combination of a compound of the invention together with parenterally or topically-applied (including inhaled) local anaesthetic agents such as lignocaine and analogues.

The compounds of the present invention may also be used in combination with anti-osteoporosis agents including hormonal agents such as raloxifene, and biphosphonates such as alendronate.

The present invention still further relates to the combination of a compound of the invention together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) Kinase inhibitors including but not limited to inhibitors of tyrosine kinases (such as Btk, Itk, Jak3 MAP examples of inhibitors might include Gefitinib, Imatinib mesylate), Serine/threonine kinases (including but not limited to inhibitors of MAP kinases such as p38, JNK, protein kinases A, B and C and IKK), and kinases involved in cell cycle regulation (such as but not limted to the cylin dependent kinases); (viii) glucose-6 phosphate dehydrogenase inhibitors; (ix) kinin-B₁- and B₂-receptor antagonists; (x) anti-gout agents, e.g., colchicine; (xi) xanthine oxidase inhibitors, e.g., allopurinol; (xii) uricosuric agents, e.g., probenecid, sulfnpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) tachykinin NK₁ and NK₃ receptor antagonists such as the group consisting of NKP-608C; SB-233412 (talnetant); and D4418; (xx) elastase inhibitors such as the group consisting of UT-77 and ZD-0892; (xxi) TNF-alpha converting enzyme inhibitors (TACE); (xxii) induced nitric oxide synthase (iNOS) inhibitors or (xxiii) chemoattractant receptor-homologous molecule expressed on TH2 cells, (such as CRTH2 antagonists) (xxiv) inhibitors of P38 (xxv) agents modulating the function of Toll-like receptors (TLR) and (xxvi) agents modulating the activity of purinergic receptors such as P2X7; (xxvii) inhibitors of transcription factors activation such as NFkB, API, and STATS.

The compounds of the invention can also be used in combination with existing therapeutic agents for the treatment of cancer. Suitable agents to be used in combination include:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab and the anti-erbb1 antibody cetuximab [C225]) , farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab, compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:
(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for m/z are given, generally only ions which indicate the parent mass are reported, and unless otherwise stated the mass ion quoted is the positive mass ion—(M+H)⁺;
(iii) the title and sub-title compounds of the examples and methods were named using the index name program from Advanced Chemistry Development Inc;
(iv) unless stated otherwise, reverse phase HPLC was conducted using a Symmetry™, NovaPak™ or Xerra™ reverse phase silica column; and

(v) the following abbreviations are used:

| | | | |
|---|---|---|---|
| Boc or BOC | tert-butoxycarbonyl | DMSO | dimethylsulfoxide |
| HPLC | high pressure liquid chromatography | aq | aqueous |
| DIPEA | Diisopropylethylamine | RT | room temperature |
| RPHPLC | Reverse phase HPLC | TFA | Trifluoroacetic acid |
| EDCI | 1-(3-Dimethylaminopropyl)-3- | HOBT | 1-hydroxybenzotriazole |
| | ethylcarbodiimide hydrochloride | | hydrate |
| DMAP | 4-Dimethylaminopyridine | Ac | CH₃C(O) |
| h | Hours | min | minutes |

| | |
|---|---|
| HATU | O-(7-azabenzotriazol-1-yl)-N,N,N′,N′-tetramethyluronium hexafluoro- |
| | phosphate |
| PyBrOP | bromo-tris-pyrrolidinophosphonium hexafluorophosphate |

### INTERMEDIATE 1

This process illustrates the preparation of 4-(3,4-dichlorophenoxy)-1-(4-piperidinylmethyl)-piperidine

a) 1,1-Dimethylethyl 4-[[4-(3,4-dichlorophenoxy)-1-piperidinyl]methyl]-1-piperidinecarboxylate

4-(3,4-Dichlorophenoxy)piperidine (1.27 g) was dissolved in tetrahydrofuran (20 mL); and then acetic acid (0.5 mL) and tert-butyl 4-formylpiperidine-1-carboxylate (1.43 g) were added to the solution. The reaction mixture was stirred at room temperature for 30 min then sodium triacetoxyborohydride (1.53 g) was added and the mixture was stirred at room temperature overnight. The reaction mixture was poured into 2 M sodium hydroxide solution (50 mL) and product was extracted with diethyl ether. The combined ether extracts were washed with brine, dried, filtered and evaporated. Crude material was purified by flash chromatography, (eluting with 979:20:1 dichloromethane:methanol:aqueous ammonia) to give the sub-title compound (2.15 g).

MS 443/445 [M+H]⁺(ES+)

¹H NMR δ (CDCl₃) 1.06 (2H, ddd), 1.45 (9H, s), 1.61-1.82 (5H, m), 1.92-1.98 (2H, m), 2.16-2.27 (4H, m), 2.65-2.73 (4H, m), 4.08 (2H, d), 4.25 (1H, dq), 6.75 (1H, dd), 6.99 (1H, d), 7.30 (1H, d).

b) 4-(3,4-Dichlorophenoxy)-1-(4-piperidinylmethyl)-piperidine

1,1-Dimethylethyl 4- {[4-(3,4-dichlorophenoxy)piperidin-1-yl]methyl}piperidine-1-carboxylate (1.0 g) was added to a mixture of 20% TFA in dichloromethane (20 mL) and the mixture was stirred at room temperature for 1 h. Solvent was removed by evaporation and 2 M sodium hydroxide solution (25 mL) was added to the residue. The product was extracted with ethyl acetate and the organic phase was washed with brine, dried, filtered and evaporated to give the title compound (0.5 g).

MS 343/345 [M+H]⁺(ES+).

¹H NMR δ (CDCl₃) 1.10 (2H, qd), 1.60 (1H, qquintet), 1.73-1.83 (4H, m), 1.90-2.01 (2H, m), 2.16-2.26 (4H, m), 2.55-2.70 (4H, m), 3.09 (2H, d), 4.24 (1H, dquintet), 6.75 (1H, dd), 6.99 (1H, d), 7.27 (1H, d).

The following Intermediates were prepared analogously from the appropriate aryloxy piperidine:

| Intermediate | Name (M + H) | ¹H NMR δ (CDCl₃) |
|---|---|---|
| 2 | 4-(2,4-Dichloro-3- | 1.13-1.27(2H, m), 1.57-1.70(1H, |
| | methylphenoxy)-1-(4- | m), 1.76-2.00(2H, m), |
| | piperidinylmethyl)-piperidine | 2.16-2.32(4H, m), 2.46(3H, s), 2.60-2.99(8H, |
| | (357/359) | m), 3.16(2H, d), 4.31(1H, quintet), |
| | | 6.75(1H, d), 7.18(1H, d) |
| 3 | 4-(4-Chloro-2- | 1.08-1.21(2H, m), 1.56-1.68(1H, |
| | methylphenoxy)-1-(4- | m), 1.73-1.86(4H, m), |
| | piperidinylmethyl)-piperidine | 1.90-1.99(2H, m), 2.16-2.31(7H, m), |
| | (322/324) | 2.57-2.69(4H, m), 3.12(2H, d), |
| | | 4.23-4.31(1H, m), 6.74(1H, d), 7.06(1H, dd), |
| | | 7.11(1H, d) |
| 4 | 4-(3,4-Dichloro-2- | (CD₃OD) 1.10-1.22(2H, m), |
| | methylphenoxy)-1-(4- | 1.66-1.85(5H, m), 1.94-2.04(2H, m), |
| | piperidinylmethyl)-piperidine | 2.22(2H, d), 2.31(3H, s), 2.32-2.41(2H, |
| | (357/359) | m), 2.59-2.72(4H, m), 3.08(2H, d), |
| | | 4.38-4.46(1H, m), 6.91(1H, d), |
| | | 7.27(1H, d) |
| 5 | 4-[(4-Fluorophenyl)methyl]-1- | (CD₃OD+DMSO) 1.19-1.32(4H, m), |
| | (4-piperidinylmethyl)- | 1.46-1.54(1H, m), 1.55-1.62(2H, |
| | piperidine | m), 1.77-1.84(1H, m), |
| | (291) | 1.85-1.93(4H, m), 2.17(2H, d), 2.51(2H, d), |
| | | 2.80-2.89(4H, m), 3.23-3.26(2H, m), |
| | | 7.01(2H, t), 7.16(2H, dd) |

### INTERMEDIATE 6

This process illustrates the preparation of 4-methoxy-1,2-benzenedicarboxylic acid 1-methyl ester

2-Bromo-5-methoxy-benzoic acid (0.5 g, 2.16 mmol) was dissolved in MeOH (10 mL) and triethylamine (5 mL) was added. It was placed in an autoclave, charged to 6 bar with carbon monoxide and heated to 90° C. for 17 h. The solvents were evaporated and the product was purified by chromatography (dichloromethane/MeOH/AcOH, 998/2/0.2) to give the title compound (200 mg).

MS 209 [M+H]⁺(ES+).

### EXAMPLE 1

This Example illustrates the preparation of 2-[[4-[[4-(3,4-dichlorophenoxy)-1-piperidinyl]methyl]-1-piperidinyl]carbonyl]-benzoic acid.

4-{[4-(3,4-Dichlorophenoxy)piperidin-1-yl]methyl}piperidine (0.24 g), triethylamine (0.107 mL) and phthalic anhydride (0.109 g) were dissolved in acetonitrile (0.5 mL) and the reaction mixture was heated in a microwave oven at 80° C. for 10 min. The solution was acidified to pH 4 by the addition of AcOH and was purified by RPHPLC (5:95 MeCN:NH₄OAc (0.1% aq) gradient to 60:40 MeCN:NH₄OAc) to provide the title compound as a white solid (0.157 g).

MS [M+H]⁺(ES+) 491/493.

¹H NMR δ (DMSO) 0.98-1.17 (2H, m), 1.49-1.67 (3H, m), 1.71-1.83 (2H, m), 1.85-1.97 (1H, m), 2.10-2.30 (4H, m), 2.60-2.75 (3H, m), 2.81-2.97 (2H, m), 3.15-3.27 (2H, m), 4.38-4.55 (2H, m), 6.97 (1H, dd), 7.22-7.27 (2H, m), 7.45-7.53 (2H, m), 7.60 (1H, td), 7.90 (1H, d).

The following Examples were prepared analogously to Example 1 from the appropriate amine and anhydride.

| Example | Name (M + H) | ¹H NMR δ (CD₃OD) |
|---|---|---|
| 2 | 2-[[4-[[4-(2,4-Dichloro-3- | 1.17-1.29(1H, m), 1.36-1.66(2H, m), |
| | methylphenoxy)-1- | 1.91-2.19(5H, m), 2.36(3H, s), |
| | piperidinyl]methyl]-1- | 2.71-2.99(4H, m), 3.07-3.19(4H, m), |
| | piperidinyl]carbonyl]-benzoic | 3.26-3.37(2H, m), 4.51-4.66(2H, m), |
| | acid | 6.91(1H, d), 7.05-7.14(1H, m), 7.19(1H, |
| | (505/507) | d), 7.30-7.42(2H, m), 7.87(1H, d) |
| 3 | 2-[[4-[[4-(3,4-Dichloro-2- | 1.30-1.51(2H, m), 1.59-1.71(1H, m), |
| | methylphenoxy)-1- | 1.78-1.89(4H, m), 1.98-2.08(2H, m), |
| | piperidinyl]methyl]-1- | 2.26-2.32(2H, m), 2.34(3H, s), |
| | piperidinyl]carbonyl]-benzoic | 2.35-2.44(2H, m), 2.67-2.76(2H, m), |
| | acid | 2.86(1H, t), 3.03(1H, t), 3.38-3.46(1H, m), |
| | (505/507) | 4.41-4.50(1H, m), 4.68(1H, d), |
| | | 6.94(1H, d), 7.17-7.26(1H, m), 7.31(1H, |
| | | d), 7.41-7.52(2H, m), 7.94(1H, d) |
| 4 | 2-[[4-[[4-(3,4-Dichlorophenoxy)- | (CD₃OD + NaOD) 1.06-1.21(1H, m), |
| | 1-piperidinyl]methyl]-1- | 1.24-1.36(2H, m), 1.43-1.68(2H, m), |
| | piperidinyl]carbonyl]-3,6- | 1.68-1.93(4H, m), 1.93-2.05(2H, m), |
| | difluoro-benzoic acid | 2.16-2.37(3H, m), 2.65-2.87(2H, m), |
| | (527/529) | 3.03-3.16(1H, m), 3.45-3.59(1H, m), |
| | | 4.32-4.41(1H, m), 4.55-4.64(1H, m), |
| | | 6.87(1H, d), 7.03-7.18(3H, m), |
| | | 7.36(1H, d) |
| 5 | 2-[[4-[[4-[(4- | 1.00-1.19(1H, m), 1.21-1.34(4H, m), |
| | Fluorophenyl)methyl]-1- | 1.41-1.54(2H, m), 1.58(2H, d), |
| | piperidinyl]methyl]-1- | 1.73-1.91(4H, m), 2.13-2.24(2H, m), |
| | piperidinyl]carbonyl]-benzoic | 2.51(2H, d), 2.72-2.83(1H, m), 2.88(2H, |
| | acid | d), 2.91-3.02(1H, m), 4.61(1H, d), |
| | (439) | 6.93-6.99(2H, m), 7.10-7.19(3H, m), |
| | | 7.36-7.45(2H, m), 7.90(1H, dd) |

### EXAMPLE 6

This Example illustrates the preparation of methyl 2-[[4-[[4-(3,4-dichlorophenoxy)-1-piperidinyl]methyl]-1-piperidinyl]carbonyl]-benzeneacetate.

To a stirred solution of 4-{[4-(3,4-dichlorophenoxy)piperidin-1-yl]methyl}piperidine (0.24 g), 2-carboxy benzeneacetic acid methyl ester (0.143 g) and diisopropylethylamine (0.27 mL) in dichloromethane at RT was added PyBrOP (0.392 g). The reaction was stirred for 16 h. The reaction mixture was diluted with 2:2:1 acetonitrile:methanol:water (5 mL) and acidified to pH 6 with acetic acid and then subjected to purification using RPHPLC (25:75 MeCN:NH₄OAc (0.1% aq) gradient to 95:5 MeCN:NH₄OAc) to provide the title compound as a white solid (0.220 g).

MS [M+H]⁺(ES+) 519/521.

¹H NMR δ(CD₃OD) 1.01-1.18 (2H, m), 1.57-1.65 (1H, m), 1.66-1.76 (2H, m), 1.77-1.86 (2H, m), 1.89-1.98 (3H, m), 2.26-2.33 (2H, m), 2.39 (2H, t), 2.69-2.79 (2H, m), 2.83-3.07 (1H, m), 3.38-3.53 (2H, m), 3.57 (3H, s), 3.62-3.82 (1H, m), 4.31-4.38 (1H, m), 4.55 (1H, d), 6.80 (1H, dd), 7.01 (1H, d), 7.10-7.20 (1H, m), 7.22-7.34 (4H, m).

### EXAMPLE 7

This Example illustrates the preparation of methyl 3-[[4-[[4-(3,4-dichlorophenoxy)-1-piperidinyl]methyl]-1-piperidinyl]carbonyl]-benzoate

To a stirred solution of 4-{[4-(3,4-dichlorophenoxy)piperidin-1-yl]methyl}piperidine (0.48 g), monomethyl isophthalate (0.261 g) and diisopropylethylamine (0.54 mL) in dichloromethane (7 mL) at RT was added PyBrOP (0.7 g). The reaction was stirred for 16 h. The reaction mixture was diluted with dichloromethane and subjected directly to flash column chromatography, eluting with 96:4 dichloromethane/methanol to leave a colourless oil (0.65 g).

MS [M+H]⁺(ES+) 505/507.

### EXAMPLE 8

This Example illustrates the preparation of methyl 2-[[4-[[4-(3,4-dichlorophenoxy)-1-piperidinyl]methyl]-1-piperidinyl]carbonyl]-4-methoxybenzoate.

4-(3,4-Dichlorophenoxy)-1-(4-piperidinylmethyl)-piperidine (343 mg), EDCI (286 mg), HOBT (135 mg), DMAP (122 mg) were dissolved in dichloromethane (20 mL) and triethylamine (0.3 mL) was added. The reaction mixture was stirred for 72 h. The solvents were evaporated and the residue was purified by RPHPLC (gradient 95%-5% aqueous ammonium acetate, 5%-95% acetonitrile) to give the title compound (30 mg).

MS [M+H]⁺(ES+) 535/537.

The following Examples were prepared analogously to Example 8 from the appropriate acids and amines.

| Example | Name (M+H) | ¹H NMR δ(CD₃OD) |
|---|---|---|
| 9 | Methyl 4-[[4-[[4-(3,4- | |
| | dichlorophenoxy)-1- | |
| | piperidinyl]methyl]-1- | |
| | piperidinyl]carbonyl]-benzoate | |
| | (505/507) | |
| 10 | 1-Methylethyl 3-[[4-[[4-(3,4- | 1.16-1.36(2H, m), 1.40(6H, dd), |
| | dichlorophenoxy)-1- | 1.69-2.07(7H, m), 2.27-2.40(4H, m), |
| | piperidinyl]methyl]-1- | 2.71-2.79(2H, m), 2.85-2.96(1H, m), |
| | piperidinyl]carbonyl]-2- | 3.06-3.17(1H, m), 3.36-3.45(1H, m), |
| | pyridinecarboxylate | 4.36-4.46(1H, m), 4.62-4.70(1H, m), |
| | (534/536) | 5.26(1H, q), 6.90(1H, dd), 7.11(1H, d), |
| | | 7.39(1H, d), 7.71(1H, dd), 7.89(1H, dd), |
| | | 8.74(1H, dd) |
| 11 | Methyl 4-chloro-2-[[4-[[4-(3,4- | |
| | dichlorophenoxy)-1- | |
| | piperidinyl]methyl]-1- | |
| | piperidinyl]carbonyl]-benzoate | |
| | (541/543) | |

### EXAMPLE 12

This Example illustrates the preparation of 2-[[4-[[4-(3,4-dichlorophenoxy)-1-piperidinyl]methyl]-1-piperidinyl]carbonyl]-benzeneacetic acid

A solution of 2-[[4-[[4-(3,4-dichlorophenoxy)-1-piperidinyl]methyl]-1-piperidinyl]carbonyl]-benzeneacetic acid methyl ester (0.188 g) and lithium hydroxide (0.046 g) in 3:1 methanol:water (2 mL) was stirred at RT for 16 h. The reaction mixture was acidified to pH 6 with acetic acid and subjected to purification using RPHPLC (5:95 MeCN:NH₄OAc (0.1% aq) gradient to 50:50 MeCN:NH₄OAc) to provide the title compound as a white solid (0.151 g).

MS [M+H]⁺(ES+) 505/507.

¹H NMR δ (CD₃OD) 1.09 (1H, qd), 1.15-1.31 (2H, m), 1.60-1.80 (3H, m), 1.80-1.93 (2H, m), 1.94-2.02 (2H, m), 2.23-2.27 (1H, m), 2.27-2.36 (2H, m), 2.66-2.76 (2H, m), 2.79-2.92 (1H, m), 2.98-3.09 (1H, m), 3.33-3.45 (1H, m), 3.44-3.62 (2H, m), 4.34-4.41 (1H, m), 4.65 (1H, d), 6.88 (1H, dd), 7.07 (1H, d), 7.09-7.20 (1H, m), 7.21-7.29 (1H, m), 7.31-7.42 (3H, m).

The following Examples were prepared analogously to Example 12 from the appropriate esters.

| Example | Name (M + H) | ¹H NMR δ |
|---|---|---|
| 13 | 3-[[4-[[4-(3,4-Dichlorophenoxy)- | (DMSO, 120° C.) 1.12(2H, q), |
| | 1-piperidinyl]methyl]-1- | 1.59-1.68(2H, m), 1.70-1.82(3H, m), |
| | piperidinyl]carbonyl]-benzoic | 1.86-1.93(2H, m), 2.22(2H, d), 2.27(2H, |
| | acid | ddd), 2.63-2.70(2H, m), 2.95(2H, t), |
| | (491/493) | 3.92-4.00(2H, m), 4.34-4.39(1H, m), |
| | | 6.93(1H, dd), 7.14(1H, d), 7.42(1H, d), |
| | | 7.50-7.57(2H, m), 7.87(1H, s), |
| | | 7.97(1H, dt), resonance for one proton |
| | | obscured |
| 14 | 3-[[4-[[4-(3,4-Dichlorophenoxy)- | (CD₃OD) 1.27-1.46(2H, m), |
| | 1-piperidinyl]methyl]-1- | 1.66-1.74(1H, m), 1.82-1.90(1H, m), |
| | piperidinyl]carbonyl]-2- | 1.95-2.11(3H, m), 2.14-2.26(2H, m), |
| | pyridinecarboxylic acid | 2.75-2.90(3H, m), 2.96-3.29(6H, m), |
| | (492/494) | 4.55-4.67(2H, m), 6.93(1H, dd), 7.17(1H, |
| | | d), 7.40(1H, d), 7.46(1H, dd), 7.65(1H, |
| | | d), 8.59(1H, d) |

### EXAMPLE 15

This Example illustrates the preparation of 2-[[4-[[4-(3,4-dichlorophenoxy)-1-piperidinyl]methyl]-1-piperidinyl]carbonyl]-4-methoxybenzoic acid

Methyl 2-[[4-[[4-(3,4-dichlorophenoxy)-1-piperidinyl]methyl]-1-piperidinyl]carbonyl]-4-methoxy-benzoate (30 mg, 5.61 mmol) was dissolved in THF (10 mL) and potassium trimethylsilanolate (500 mg) was added. The mixture was stirred at room temparature for 2 h and was then acidified using AcOH. The volatiles were evaporated and the residue was redissolved in MeOH and was purified by RPHPLC (gradient 95%-0.5% aqueous ammonium acetate, 5%-95% acetonitrile) to give the title compound (15 mg).

MS [M+H]⁺(ES+) 521/523.

¹H NMR δ(CD₃OD+NaOD) 1.06-1.21 (1H, m), 1.24-1.36 (2H, m), 1.43-1.68 (2H, m), 1.68-1.93 (4H, m), 1.93-2.05 (2H, m), 2.16-2.37 (3H, m), 2.65-2.87 (2H, m), 3.03-3.16 (1H, m), 3.45-3.59 (1H, m), 4.32-4.41 (1H, m), 4.55-4.64 (1H, m), 6.87 (1H, d), 7.03-7.18 (3H, m), 7.36 (1H, d).

The following Examples were prepared analogously to Example 15 from the appropriate esters which are either reported above or may prepared by analogous routes. In Example 18 the product crystallized at the end of the reaction and was collected by filtration. The product was dissolved in aqueous sodium hydroxide and the water was evaporated to leave the product as the sodium salt.

| Example | Name (M + H) | ¹H NMR δ |
|---|---|---|
| 16 | 4-[[4-[[4-(3,4-Dichlorophenoxy)- | (CD₃OD + NaOD) 1.15-1.39(2H, m), |
| | 1-piperidinyl]methyl]-1- | 1.73-1.95(2H, m), 1.96-2.06(2H, m), |
| | piperidinyl]carbonyl]-benzoic | 2.07-2.24(3H, m), 2.83(2H, d), |
| | acid | 2.86-2.96(1H, m), 2.98-3.08(2H, m), |
| | (491/493) | 3.08-3.24(3H, m), 3.60-3.70(1H, m), |
| | | 4.58-4.69(2H, m), 6.95(1H, dd), 7.19(1H, |
| | | d), 7.38-7.44(3H, m), 8.01(2H, d) |
| 17 | 2-[[4-[[4-(3,4-Dichlorophenoxy)- | (CD₃OD) 0.94-1.36(2H, m), |
| | 1-piperidinyl]methyl]-1- | 1.37-1.67(2H, m), 1.69-1.85(3H, m), |
| | piperidinyl]carbonyl]-4- | 1.94-2.03(2H, m), 2.17-2.35(4H, m), |
| | methylbenzoic acid | 2.37(3H, d), 2.67-2.75(2H, m), |
| | (505/507) | 2.76-2.86(1H, m), 2.91-3.04(1H, m), |
| | | 3.34-3.44(1H, m), 4.33-4.41(1H, m), 4.62(1H, |
| | | d), 6.87(1H, dd), 6.90-7.07(1H, m), |
| | | 7.08(1H, d), 7.19-7.27(1H, m), |
| | | 7.36(1H, d), 7.71-7.83(1H, m) |
| 18 | 4-Chloro-2-[[4[[4-(3,4- | (CD₃OD + NaOD) 0.89-1.89(9H, m), |
| | dichlorophenoxy)-1- | 2.13-2.27(4H, m), 2.58-2.79(3H, m), |
| | piperidinyl]methyl]-1- | 2.85-2.97(1H, m), 3.23-3.31(1H, m), |
| | piperidinyl]carbonyl]-benzoic | 4.24-4.33(1H, m), 4.46-4.55(1H, m), |
| | acid sodium salt | 6.78(1H, dd), 6.98(1H, d), |
| | (527/529) | 7.02-7.12(1H, m), 7.25-7.33(2H, m), 7.79(1H, |
| | | d) |

### EXAMPLE 19

This Example illustrates the preparation of 4-[[4-(3,4-dichlorophenoxy)-1-piperidinyl]methyl]-1-[4-hydroxy-3-(methylsulfonyl)benzoyl]-piperidine.

a) 4-[[4-(3,4-Dichlorophenoxy)-1-piperidinyl]methyl]-1-[4-methoxy-3-(methylsulfonyl)benzoyl]-piperidine

To a stirred solution of 4-{[4-(3,4-dichlorophenoxy)piperidin-1-yl]methyl}piperidine (0.256 g), 4-methoxy-3-(methylsulfonyl)benzoic acid (WO 98/41598; 0.18 g) and diisopropylethylamine (0.286 mL) in dichloromethane (3 mL) at RT was added PyBrOP (0.417 g). The reaction was stirred for 16 h. The reaction mixture was diluted with 1:1 acetonitrile/methanol (5 mL) and acidified to pH 6 with acetic acid and subjected to purification using RPHPLC (5:95 MeCN:NH₄OAc (0.1% aq) gradient to 95:5 MeCN:NH₄OAc) to provide the sub-title compound as a white solid (0.290 g).

MS [M+H]⁺(ES+) 555/557.

¹H NMR δ(CD₃OD) 1.15-1.32 (2H, m), 1.78-1.91 (4H, m), 1.97-2.11 (3H, m), 2.50 (2H, d), 2.56-2.66 (2H, m), 2.87-2.95 (3H, m), 3.11-3.21 (1H, m), 3.25 (3H, s), 3.70-3.84 (1H, m), 4.06 (3H, s), 4.41-4.53 (1H, m), 4.50-4.67 (1H, m), 6.91 (1H, dd), 7.13 (1H, d), 7.35 (1H, d), 7.39 (1H, d), 7.75 (1H, dd), 7.95 (1H, d).

b) 4-[[4-(3,4-Dichlorophenoxy)-1-piperidinyl]methyl]-1-[4-hydroxy-3-(methylsulfonyl)benzoyl]-piperidine

The following reaction was performed in duplicate. A solution of 4-[[4-(3,4-dichlorophenoxy)-1-piperidinyl]methyl]-1-[4-methoxy-3-(methylsulfonyl)benzoyl]-piperidine (0.125 g) and sodium ethane thiolate (0.002 g) in DMF (4 mL) at RT was heated in a microwave oven at 150° C. for 25 min. The DMF was removed in vacuo, the residue was diluted with 1:1 acetonitrile/methanol (5 mL) and acidified to pH 6 with acetic acid. Purification using RPHPLC (5:95 MeCN:NH₄OAc (0.1% aq) gradient to 50:50 MeCN:NH₄OAc) provided the title compound as a white solid (0.014 g).

MS [M+H]⁺(ES+) 541/543.

¹H NMR δ(CD₃OD) 1.06-1.21 (3H, m), 1.69-1.81 (4H, m), 1.87-2.01 (3H, m), 2.41 (2H, d), 2.47-2.56 (2H, m), 2.78-2.86 (3H, m), 2.88-3.11 (2H, m), 3.17 (3H, s), 4.35-4.42 (1H, m), 6.81 (1H, dd), 6.94 (1H, d), 7.03 (1H, d), 7.29 (1H, d), 7.48 (1H, dd), 7.77 (1H, d).

### EXAMPLE 20

### Pharmacological Analysis: Calcium flux [Ca²⁺]ᵢ Assay

Human Eosinophils

Human eosinophils were isolated from EDTA anticoagulated peripheral blood as previously described (Hansel et al., J Immunol. Methods, 1991, 145, 105-110). The cells were resuspended (5×10⁶ ml⁻¹) and loaded with 5 μM FLUO-3/AM+Pluronic F127 2.2 μl/ml (Molecular Probes) in low potassium solution (LKS; NaCl 118 mM, MgSO₄ 0.8 mM, glucose 5.5 mM, Na₂CO₃ 8.5 mM, KCl 5 mM, HEPES 20 mM, CaCl₂ 1.8 mM, BSA 0.1%, pH 7.4) for one hour at room temperature. After loading, cells were centrifuged at 200 g for 5 min and resuspended in LKS at 2.5×10⁶ ml⁻¹. The cells were then transferred to 96 well FLIPr plates (Poly-D-Lysine plates from Becton Dickinson pre-incubated with 5 μM fibronectin for two hours) at 25 μl/well. The plate was centrifuged at 200 g for 5 min and the cells were washed twice with LKS (200 μl; room temperature).

A compound of the Examples was pre-dissolved in DMSO and added to a final concentration of 0.1% (v/v) DMSO. Assays were initiated by the addition of an A₅₀ concentration of eotaxin and the transient increase in fluo-3 fluorescence (1_{EX}=490 nm and 1_{Em}=520 nm) monitored using a FLIPR (Fluorometric Imaging Plate Reader, Molecular Devices, Sunnyvale, U.S.A.).

Compounds of the Examples were found to be antagonists if the increase in fluorescence induced by eotaxin (a selective CCR3 agonist) was inhibited in a concentration dependent manner. The concentration of antagonist required to inhibit the fluorescence by 50% can be used to determine the IC₅₀ for the antagonist at the CCR3 receptor.

### EXAMPLE 21

Human Eosinophil Chemotaxis

Human eosinophils were isolated from EDTA anticoagulated peripheral blood as previously described (Hansel et al., J. Immunol. Methods, 1991, 145, 105-110). The cells were resuspended at 10×10⁶ ml⁻¹ in RPMI containing 200 IU/ml penicillin, 200 μg/ml streptomycin sulfate and supplemented with 10% HIFCS, at room temperature.

Eosinophils (700 μl) were pre-incubated for 15 mins at 37° C. with 7 μl of either vehicle or compound (100× required final concentration in 10% DMSO). The chemotaxis plate (ChemoTx, 3 μm pore, Neuroprobe) was loaded by adding 28 μl of a concentration of eotaxin 0.1 to 100 nM (a selective CCR3 agonist over this concentration range) containing a concentration of a compound according to the Examples or solvent to the lower wells of the chemotaxis plate. The filter was then placed over the wells and 25 μl of eosinophil suspension were added to the top of the filter. The plate was incubated for 1 hr at 37° C. in a humidified incubator with a 95% air/5% CO₂ atmosphere to allow chemotaxis.

The medium, containing cells that had not migrated, was carefully aspirated from above the filter and discarded. The filter was washed once with phosphate buffered saline (PBS) containing 5 mM EDTA to remove any adherent cells. Cells that had migrated through the filter were pelleted by centrifugation (300× g for 5 mins at room temperature) and the filter removed and the supernatant transferred to each well of a 96-well plate (Costar. The pelleted cells were lysed by the addition of 28 μl of PBS containing 0.5% Triton x100 followed by two cycles of freeze/thawing. The cell lysate was then added to the supernatant. The number of eosinophils migrating was quantified according to the method of Strath et al., J. Immunol. Methods, 1985, 83, 209 by measuring eosinophil peroxidase activity in the supernatant.

Compounds of the Examples were found to be antagonists of eotaxin mediated human eosinophil chemotaxis if the concentration response to eotaxin was shifted to the right of the control curve. Measuring the concentration of eotaxin required to give 50% chemotaxis in the presence or absence of compounds enables the apparent affinity of the compounds at CCR3 to be calculated.

### EXAMPLE 22

Guinea-Pig Isolated Trachea

(See for example, Harrison, R. W. S., Carswell, H. & Young, J. M. (1984) European J. Pharmacol., 106, 405-409.)

Male albino Dunkin-Hartley guinea-pigs (250 g) were killed by cervical dislocation and the whole trachea removed. After clearing the adherent connective tissue, the trachea was cut into six ring segments each three cartilage bands wide and then suspended in 20 ml organ baths containing Krebs-Henseleit solution of the following composition (mM): NaCl 117.6, NaH₂PO₄ 0.9, NaHCO₃ 25.0, MgSO₄ 1.2, KCl 5.4, CaCl₂ 2.6 and glucose 11.1. The buffer was maintained at 37° C. and gassed with 5% CO₂ in oxygen. Indomethacin (2.8 μM) was added to the Krebs solution to prevent development of smooth muscle tone due to the synthesis of cyclo-oxygenase products. The tracheal rings were suspended between two parallel tungsten wire hooks, one attached to an Ormed beam isometric force transducer and the other to a stationary support in the organ bath. Changes in isometric force were recorded on 2-channel Sekonic flat bed chart recorders.

Experimental Protocols

At the beginning of each experiment a force of 1 g was applied to the tissues and this was reinstated over a 60 minute equilibration period until a steady resting tone was achieved. Subsequently, a cumulative histamine concentration effect (E/[A]) curve was constructed at 0.5 log₁₀ unit increments, in each tissue. The tissues were then washed and approximately 30 minutes later, test compound or vehicle (20% DMSO) was added. Following an incubation period of 60 minutes a second E/[A] curve was performed to histamine.

Contraction responses were recorded as a percentage of the first curve maximum.

Data Analysis

Experimental E/[A] curve data were analysed for the purposes of estimating the potencies (p[A₅₀] values) of histamine in the absence and presence of the test compound. Affinity (pA₂) values of test compounds were subsequently calculated using the following equation:
log(r−1)=log[B]+pA₂
where r=[A]₅₀ in presence of test compound/[A]₅₀ in absence of antagonist and [B] is the concentration of test compound. Compounds of the Examples were found to be H1 antagonists.

### EXAMPLE 23

Histamine H1 receptor binding activity of compounds of the invention was assessed by competition displacement of 1 nM [3H]-pyrilamine (Amersham, Bucks, Product code TRK 608, specific activity 30 Ci/mmol) to 2 μg membranes prepared from recombinant CHO-K1 cells expressing the human H1 receptor (Euroscreen SA, Brussels, Belgium, product code ES-390-M) in assay buffer (50 mM Tris pH 7.4 containing 2 mM MgCl₂, 250 mM sucrose and 100 mM NaCl) for 1 hour at room temperature.

| Example | H1 pKi/[1328_S] |
|---|---|
| 1 | 6.5 |
| 2 | 7.2 |
| 3 | 6.7 |
| 12 | 6.6 |
| 19 | 7.5 |

1. A compound of formula (I): 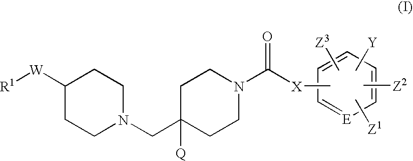
wherein:
E is CH or N;
Q is hydrogen or hydroxy;
W is CH₂, O or NR²;
X is a bond, CH₂ or CH₂O;
Y is OH, CO₂R³, SO₃H, CH₂CO₂R³, CH₂SO₃H, OCH₂CO₂R³ or OCH₂SO₃H;
Z¹, Z², Z³ are, independently, hydrogen, halogen, cyano, nitro, hydroxy, NR⁴R⁵, C₁₋₆ alkyl (optionally substituted with halogen), C₁₋₆ alkoxy (optionally substituted with halogen), S(O)ₚ(C₁₋₆ alkyl), S(O)_{q}CF₃ or S(O)₂NR⁶R⁷;
R¹ is phenyl optionally substituted by halogen, cyano, C₁₋₄ alkyl, C₁₋₄ haloalkyl, C₁₋₄ alkoxy or C₁₋₄ haloalkoxy;
R² is hydrogen or C₁₋₄ alkyl;
R³ is hydrogen, C₁₋₆ alkyl or benzyl;
p and q are, independently, 0, 1 or 2;
R⁴, R⁵, R⁶ and R⁷ are, independently, hydrogen, C₁₋₆ alkyl (optionally substituted by halogen, hydroxy or C₃₋₁₀ cycloalkyl), CH₂(C₂₋₅ alkenyl), phenyl (itself optionally substituted by halogen, hydroxy, nitro, NH_{2,} NH(C₁₋₄ alkyl), N(C₁₋₄ alkyl)₂ (and these alkyl groups may join to form a ring as described for R⁴ and R⁵ below), S(O)₂(C₁₋₄ alkyl), S(O)₂NH₂, S(O)₂NH(C₁₋₄ alkyl), S(O)₂N(C₁₋₄ alkyl)2 (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂ (and these alkyl groups may join to form a ring as described for R⁴ and R⁵ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁴ and R⁵ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁴ and R⁵ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
alternatively NR⁴R⁵ or NR⁶R⁷ may, independently, form a 4-7 membered heterocyclic ring, azetidine, pyrrolidine, piperidine, azepine, morpholine or piperazine, the latter optionally substituted by C₁₋₄ alkyl on the distal nitrogen;
or an N-oxide thereof; or a pharmaceutically acceptable salt thereof; or a solvate thereof.

2. A compound of formula (I) as claimed in claim 1 wherein W is O.

3. A compound of formula (I) as claimed in claim 1 wherein E is CH.

4. A compound of formula (I) as claimed in claim 1, wherein R¹ is phenyl optionally substituted with halogen, C₁₋₄ alkyl or C₁₋₄ alkoxy.

5. A compound of formula (I) as claimed in claim 1, wherein Y is CO₂H, CO₂(C₁₋₄ alkyl), CH₂CO₂H or OH.

6. A compound of formula (I) as claimed in claim 1, wherein Z¹, Z² and Z³ are, independently, hydrogen, halogen, cyano, C₁₋₄ alkyl, C₁₋₄ alkoxy, CF₃, OCF₃, S(O)₂(C₁₋₄ alkyl) or S(O)₂NH₂.

7. A process for preparing a compound of formula (I) as claimed in claim 1, the process comprising:
a. when Y is CO₂H, CH₂CO₂H or OCH₂CO₂H, said Y group being ortho to the group X, acylating a compound of formula (II): 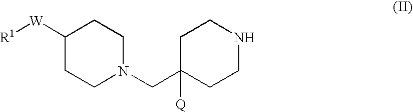
via the ring opening of an anhydride of formula (III): 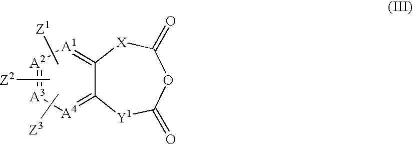
wherein one of A¹, A², A³ and A⁴ is CH or N; the other three of A¹, A², A³ and A⁴ are carbon and each of the three carries Z¹, Z² or Z³, there being only one of each of Z¹, Z² and Z³; X is as defined in claim 1; and Y¹ is a bond, CH₂ or OCH₂; in the presence of a suitable tertiary amine, in a suitable solvent at an elevated temperature;
b. when Y is CO₂R³, CH₂CO₂R³ or OCH₂CO₂R³ and R³ is not hydrogen, coupling a compound of formula (II) with a compound of formula (IV): 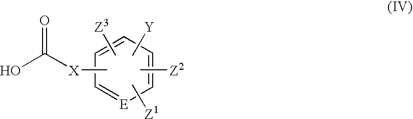
either going via the acid chloride of the compound of formula (IV) or by using a coupling reagent;
c. when X is a bond and Y is CO₂R³, carbonylating a compound of formula (V): 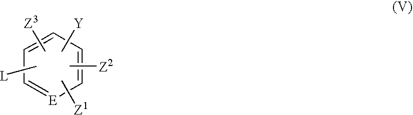
wherein L is chloro, bromo, iodo or O-triflate, and then quenching the product so formed with a compound of formula (II);
d. when X is a bond, Y is CO₂R³, R³ is not hydrogen, and R¹ does not have a chloro, bromo or iodo substituent,
i. coupling a compound of formula (II) with an acid of formula (VI): 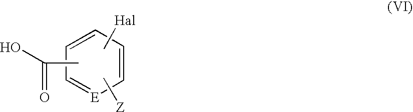
wherein Hal is chloro, bromo or iodo;
ii. carbonylating the compound so formed; and then,
iii. quenching the product so formed with a C₁₋₆ aliphatic alcohol or benzylalcohol; OR
e. when Y is or includes a CO₂R³ group:
i. when R³ is hydrogen said compound can be converted to a compound of the invention where R³ is not hydrogen by a standard esterification method; or
ii. when R³ is not hydrogen said compound can be converted to a compound of the invention where R³ is hydrogen by a standard ester hydrolysis method.

8. A pharmaceutical composition which comprises a compound of the formula (I), or a pharmaceutically acceptable salt thereof or solvate thereof as claimed in claim 1, and a pharmaceutically acceptable adjuvant, diluent or carrier.

9-10. (canceled)

11. A method of treating a chemokine mediated disease state in a mammal suffering from, or at risk of, said disease, which comprises administering to said mammal a therapeutically effective amount of a compound of formula (I), or a pharmaceutically acceptable salt thereof or solvate thereof as claimed in claim 1.

12. A compound of formula (I) as claimed in claim 2, wherein E is CH.

13. A compound of formula (I) as claimed in claim 2, wherein R¹ is phenyl optionally substituted with halogen, C₁₋₄ alkyl or C₁₋₄ alkoxy.

14. A compound of formula (I) as claimed in claim 3 wherein R¹ is phenyl optionally substituted with halogen, C₁₋₄ alkyl or C₁₋₄ alkoxy.

15. A compound of formula (I) as claimed in claim 2, wherein Y is CO₂H, CO₂(C₁₋₄ alkyl), CH₂CO₂H or OH.

16. A compound of formula (I) as claimed in claim 3, wherein Y is CO₂H, CO₂(C₁₋₄ alkyl), CH₂CO₂H or OH.

17. A compound of formula (I) as claimed in claim 4, wherein Y is CO₂H, CO₂(C₁₋₄ alkyl), CH₂CO₂H or OH.

18. A compound of formula (I) as claimed in claim 2, wherein Z¹, Z² and Z³ are, independently, hydrogen, halogen, cyano, C₁₋₄ alkyl, C₁₋₄ alkoxy, CF₃, OCF₃, S(O)₂(C₁₋₄ alkyl) or S(O)₂NH₂.

19. A compound of formula (I) as claimed in claim 3, wherein Z¹, Z² and Z³ are, independently, hydrogen, halogen, cyano, C₁₋₄ alkyl, C₁₋₄ alkoxy, CF₃, OCF₃, S(O)₂(C₁₋₄ alkyl) or S(O)₂NH₂.

20. A compound of formula (I) as claimed in claim 4, wherein Z¹, Z² and Z³ are, independently, hydrogen, halogen, cyano, C₁₋₄ alkyl, C₁₋₄ alkoxy, CF₃, OCF₃, S(O)₂(C₁₋₄ alkyl) or S(O)₂NH₂.

21. A compound of formula (I) as claimed in claim 5, wherein Z¹, Z² and Z³ are, independently, hydrogen, halogen, cyano, C₁₋₄ alkyl, C₁₋₄ alkoxy, CF₃, OCF₃, S(O)₂(C₁₋₄ alkyl) or S(O)₂NH₂.
